(19) United States
(12) Patent Application Publication
(10) Pub. No.: US 2022/0280137 A1
(43) Pub. Date: Sep. 8, 2022

(54) DATA PROCESSING METHOD FOR ULTRASONIC IMAGING SYSTEM, ULTRASONIC IMAGING SYSTEM AND STORAGE MEDIUM

(71) Applicant: BOE TECHNOLOGY GROUP CO., LTD., Beijing (CN)

(72) Inventors: Jijing HUANG, Beijing (CN); Zhiming YANG, Beijing (CN); Zongmin LIU, Beijing (CN); Qiong WU, Beijing (CN); Dawei TANG, Beijing (CN)

(21) Appl. No.: 17/630,244

(22) PCT Filed: Mar. 25, 2021
(86) PCT No.: PCT/CN2021/083027

§ 371 Date: Jan. 26, 2022

(30) Foreign Application Priority Data
Mar. 31, 2020 (CN) 202010244551.8

(51) Int. Cl. A61B 8/08; A61B 8/00

(52) U.S. Cl. CPC A61B 8/5207; A61B 8/56

(57) ABSTRACT

Provided are an ultrasonic imaging system, a data processing method for the ultrasonic imaging system and a storage medium. The data processing method for the ultrasonic imaging system includes: acquiring array element data of each of a plurality of array elements in an ultrasonic transducer array; determining one of the plurality of array elements to be a reference array element and the other array elements except the reference array element among the plurality of array elements to be to-be-compensated array elements, and determining interpolation points of the to-be-compensated array elements according to a scanning position and an acquisition moment of each array element data of the reference array element; and performing data compensation on the determined interpolation points to obtain interpolation data.

### CROSS-REFERENCE TO RELATED APPLICATIONS

The present disclosure claims the priority to the Chinese Patent Application No. 202010244551.8 filed with the CNIPA on Mar. 31, 2020, the entire contents of which are incorporated herein by reference.

### TECHNICAL FIELD

The present disclosure relates to the technical field of data processing, and in particular, to a data processing method for an ultrasonic imaging system, an ultrasonic imaging system and a storage medium.

### BACKGROUND

Ultrasonic imaging is a technique of scanning a human body with ultrasonic beams, and receiving and processing reflected signals to obtain an image of a tissue or an organ in the human body. Current ultrasonic imaging system generally adopts a multi-element ultrasonic probe, in which a plurality of array elements of the ultrasonic probe are excited by electrical signals to generate ultrasonic waves and form transmitting beams into a human body, and then receives ultrasonic echo signals scattered or reflected from an tissue or an organ in the human body through the plurality of array elements receive, and analyzes and processes the received ultrasonic echo signals through beam synthesis, dynamic filtering, envelope detection, logarithmic compression and the like to obtain an image of the tissue or organ in the human body.

### SUMMARY

In one aspect, an embodiment of the present disclosure provides a data processing method for an ultrasonic imaging system, including:

acquiring a plurality of pieces of array element data of each of a plurality of array elements in an ultrasonic transducer array;
determining one of the plurality of array elements to be a reference array element and the other array elements except the reference array element among the plurality of array elements to be to-be-compensated array elements;
determining interpolation points of the to-be-compensated array elements according to a scanning position and an acquisition moment of each array element data of the reference array element; and
performing data compensation on the determined interpolation points to obtain interpolation data.

In another aspect, an embodiment of the present disclosure provides an ultrasonic imaging system, including: an ultrasonic transducer array and an ultrasonic receiving circuit;

the ultrasonic transducer array includes a plurality of array elements; and
the ultrasonic receiving circuit is connected to and communicates with each of the plurality of array elements, and is configured to receive ultrasonic echo signals acquired by the plurality of array elements as array element data and perform the data processing method for the ultrasonic imaging system described herein.

In still another aspect, an embodiment of the present disclosure provides a computer-readable storage medium having a computer program stored thereon. When the computer program is executed by a processor, the data processing method for the ultrasonic imaging system described herein is implemented.

### BRIEF DESCRIPTION OF DRAWINGS

The above and/or additional aspects and advantages of the present disclosure will become apparent from and can be easily understood according to the following description of the embodiments with reference to the drawings. In the drawings:

FIG. 1 is a block diagram of an ultrasonic imaging system according to an embodiment of the present disclosure;

FIG. 2 is a block diagram of another ultrasonic imaging system according to an embodiment of the present disclosure;

FIG. 3 is a schematic diagram illustrating a structure of an ultrasonic transducer array and a positional relationship between the ultrasonic transducer array and an acquisition centerline according to an embodiment of the present disclosure;

FIG. 4 is a block diagram of an ultrasonic receiving circuit according to an embodiment of the present disclosure;

FIG. 5 is a block diagram of another ultrasonic receiving circuit according to an embodiment of the present disclosure;

FIG. 6 is a block diagram of still another ultrasonic receiving circuit according to an embodiment of the present disclosure;

FIG. 7 is a flowchart illustrating a data processing method for an ultrasonic imaging system according to an embodiment of the present disclosure;

FIG. 8 is a schematic diagram of a change curve of ratios of acoustic path differences of an array element 1 to acoustic path differences of an array element 4 with scan depths according to an embodiment of the present disclosure; and

FIG. 9 is a schematic diagram illustrating a principle of data compensation according to an embodiment of the present disclosure.

### DETAIL DESCRIPTION OF EMBODIMENTS

The present disclosure will be described in detail below, the examples of the embodiments of the present disclosure are illustrated in the drawings, and the same or similar reference numerals refer to the same or similar elements, or elements having the same or similar functions throughout the present disclosure. In addition, if the detailed descriptions of known technology are not necessary to the illustrated features of the present disclosure, the detailed descriptions are omitted. The embodiments described below with reference to the drawings are illustratively and only used to explain the present disclosure, and should not be understood to limit the present disclosure.

It should be understood by those of ordinary skill in the art that, unless otherwise defined, all terms (including technical and scientific terms) used herein have the same meaning as commonly understood by those of ordinary skill in the art. It should be further understood that terms, such as those defined in commonly used dictionaries, should be interpreted as having a meaning that is consistent with a meaning in the context of the existing technology art, and should not be interpreted in an idealized or overly formal sense unless expressly so defined herein.

It should be understood by those of ordinary skill in the art that, unless expressly stated, “a”, “one”, and “the” used here and indicating a singular form may indicate a plural form. It should be further understood that the term “comprise” used herein indicates the presence of the described features, integers, steps, operations, elements and/or components, but does not exclude the presence or addition of one or more other features, integers, steps, operations, elements, components and/or combinations thereof. It should be understood that when an element is “connected” or “coupled” to another element, the element may be connected or coupled to the other element directly or through an intermediate element. Further, the term “connect” or “couple” used herein may include wireless connection or wireless coupling. The term “and/or” used herein includes all or any one of one or more of associated listed items, and all combinations of the one or more of associated listed items.

In the related art, in existing ultrasonic imaging processes, array element data acquired by different array elements cannot meet a requirement of beam synthesis due to the problem of misalignment therebetween, and accuracy of ultrasonic imaging is affected if the data from the different array elements are directly synthesized without being processed at all.

According to the embodiments of the present disclosure, on the basis that a reference array element is determined, an interpolation point of a to-be-compensated array element can be determined according to the reference array element, and interpolation data of the to-be-compensated array element can be determined based on the interpolation point according to a position of an interpolation point adjacent to the interpolation point and array element data of the adjacent interpolation point, so that compensation for array element data of the to-be-compensated array element can be achieved, each to-be-compensated array element has the same amount of data as the reference array element within the same distance on a scanning line, and the array element data of each to-be-compensated array element can be aligned with that of the reference array element. Thus, the array element data of each array element can meet the requirement of beam synthesis, and the accuracy of ultrasonic imaging can be improved.

The technical solutions of the present disclosure and how to solve the above technical problem thereby are illustrated below in detail by specific embodiments.

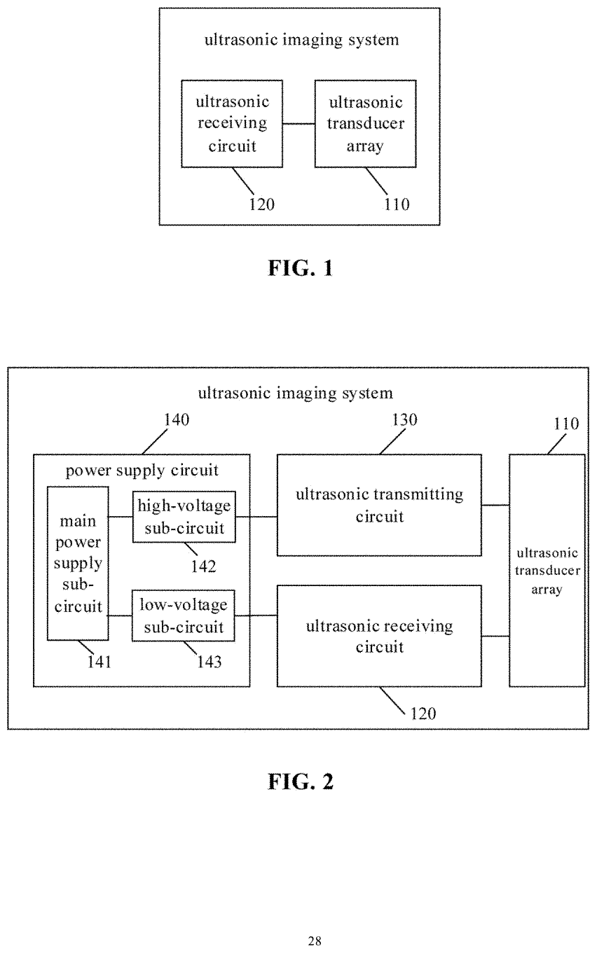

FIG. 1 is a block diagram of an ultrasonic imaging system according to an embodiment of the present disclosure. As shown in FIG. 1, in the embodiment, the ultrasonic imaging system includes: an ultrasonic transducer array 10 and an ultrasonic receiving circuit 120, and the ultrasonic transducer array 110 includes a plurality of ultrasonic transducer array elements (hereinafter referred to as “array elements”).

The ultrasonic receiving circuit 120 is connected to and communicates with each array element and is configured to receive ultrasonic echo signals acquired by the plurality of array elements as array element data and perform a data processing method for the ultrasonic imaging system provided by the embodiment of the present disclosure. The data processing method will be described in detail later in the description.

FIG. 2 is a block diagram of another ultrasonic imaging system according to an embodiment of the present disclosure. As shown in FIG. 2, in the present embodiment, the ultrasonic imaging system further includes: an ultrasonic transmitting circuit 130 and a power supply circuit 140.

The ultrasonic transmitting circuit 130 is connected to and communicates with each array element and is configured to generate an electrical signal and excite the plurality of array elements with the electrical signal to transmit ultrasonic waves. The power supply circuit 140 is electrically connected to the ultrasonic receiving circuit 120 and the ultrasonic transmitting circuit 130 (for example, through power cables) respectively, and is configured to supply power to the ultrasonic receiving circuit 120 and the ultrasonic transmitting circuit 130, and supply power to the ultrasonic transducer array 110 through the ultrasonic receiving circuit 120 and the ultrasonic transmitting circuit 130.

In an embodiment, the ultrasonic imaging system further includes: a display device, which is connected to and communicates with the ultrasonic receiving circuit 120 and is configured to display data processed by the ultrasonic receiving circuit 120 according to the data processing method for the ultrasonic imaging system provided by the embodiments of the present disclosure.

In an embodiment, the ultrasonic transducer array 110 may be an ultrasonic probe, but a type of the ultrasonic probe is not limited in the embodiments of the present disclosure. It should be understood that the technical solutions of the embodiments of the present disclosure are applicable to various ultrasonic probes.

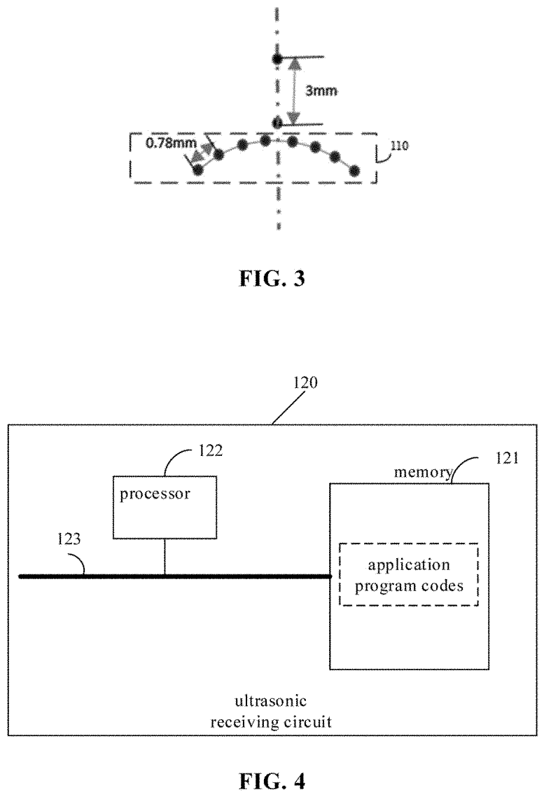

FIG. 3 is a schematic diagram illustrating a structure of an ultrasonic transducer array and a positional relationship between the ultrasonic transducer array and an acquisition centerline according to an embodiment of the present disclosure. As shown in FIG. 3, in the present embodiment, the ultrasonic transducer array 110 may be an 80-element convex array probe including eight array elements (8 dots on the curve in FIG. 3), four array elements and the other four array elements are symmetrically disposed with respect to a scanning line (the dashed line in FIG. 3, and also called an acquisition centerline), and the eight array elements may be arranged at a fixed interval, for example, at the interval of 0.78 mm shown in FIG. 3, or at an interval of other values, but the interval is not limited in the embodiments of the present disclosure.

FIG. 4 is a block diagram of an ultrasonic receiving circuit according to an embodiment of the present disclosure. As shown in FIG. 4, in the present embodiment, the ultrasonic receiving circuit 120 includes: a memory 121 and a processor 122, which are electrically connected to each other, for example, through a bus 123. The memory 121 has a computer program stored thereon, and the computer program may be executed by the processor 122 to implement the data processing method for the ultrasonic imaging system provided by the embodiments of the present disclosure.

In an embodiment, the memory 121 may be further configured to store array element data of the plurality of array elements, interpolation points and interpolation data obtained according to the data processing method for the ultrasonic imaging system provided by the embodiments of the present disclosure, and data obtained after compensation for the array element data.

In an embodiment, in a case where the ultrasonic transducer array 110 is an 80-element convex array probe, the ultrasonic receiving circuit 120 may include fourteen memories 121, with six memories 121 configured to store array element data of six to-be-compensated array elements (considering the symmetry of the array elements, two reference array elements may exist) respectively, the seventh memory 121 configured to store the interpolation points and the interpolation data obtained according to the data processing method for the ultrasonic imaging system provided by the embodiments of the present disclosure, the eighth memory 121 configured to store array element data of the reference array elements, and the remaining six memories 121 configured to store array element data obtained after compensation for the six to-be-compensated array elements, respectively.

The seventh memory 121 and the eighth memory 121 described in the embodiment of the present disclosure are mainly used to distinguish between the different memories 121, and are not intended to define an order or sequence numbers of the memories 121.

In an embodiment, the processor 122 may include six multiplier circuits configured to call the array element data of the six to-be-compensated array elements from the six memories 121 respectively, and weight the array element data, interpolation coefficients and correction coefficients of the six to-be-compensated array elements, so as to achieve compensation for the array element data.

In an embodiment, the memory 121 may be a Read-Only Memory (ROM) or other types of static storage devices capable of storing static information and instructions, or a Random Access Memory (RAM) or other types of dynamic storage devices capable of storing information and instructions. In an embodiment, the memory 121 may also be an Electrically Erasable Programmable Read-Only Memory (EEPROM), a Compact Disc Read-Only Memory (CD-ROM) or other optical discs (including a compact disc, a laser disc, an optical disc, a digital versatile disc and a Blu-ray disc), a magnetic disk or other magnetic storage devices, or any other medium that can be used to carry or store desired program codes in the form of instructions or data structures and be accessed by a computer, but the present disclosure is not limited thereto.

In an embodiment, the processor 122 may be a Central Processing Unit (CPU), a general-purpose processor, a Digital Signal Processor (DSP), an Application Specific Integrated Circuit (ASIC), a Field-Programmable Gate Array (FPGA) or other programmable logic devices, transistor logic devices, hardware components or any combination thereof. The processor 122 may implement or execute various illustrative logical blocks, modules and circuits described herein. The processor 122 may also be a combination which performs computing functions, such as a combination including one or more microprocessor, and a combination of DSP and microprocessor.

In an embodiment, the bus 123 may include a path for transferring information among the above components. The bus 123 may be a Peripheral Component Interconnect (PCI) bus or an Extended Industry Standard Architecture (EISA) bus. The bus may be an address bus, a data bus, a control bus, etc. For convenience of description, the bus is represented by one thick line alone in FIG. 4, which does not mean that there is only one bus or only one type of bus.

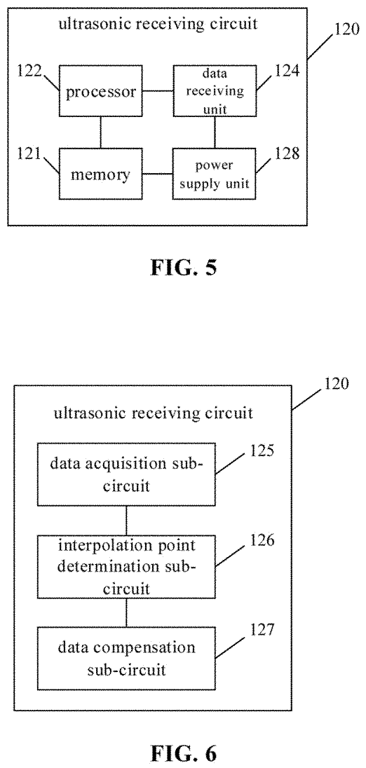

FIG. 5 is a block diagram of another ultrasonic receiving circuit according to an embodiment of the present disclosure. As shown in FIG. 5, in the embodiment, in addition to the memory 121 and the processor 122, the ultrasonic receiving circuit 120 may further include: a data receiving unit 124 and a power supply unit 125. The data receiving unit 124 are connected to and communicates with the processor 122 and all the array elements of the ultrasonic transducer array 110 respectively, and the power supply unit 125 is electrically connected to the memory 121, the processor 122, the data receiving unit 124 and the power supply circuit 140.

The data receiving unit 124 may be configured to receive ultrasonic echo signals from the array elements in the ultrasonic transducer array 110 under the control of the processor 122, amplify the ultrasonic echo signals and then feed the amplified ultrasonic echo signals to the processor 122. The power supply unit 125 may be configured to convert a voltage output from the power supply circuit 140 into voltages needed by the memory 121, the processor 122 and the data receiving unit 124 and supply power to the memory 121, the processor 122 and the data receiving unit 124 respectively.

The data receiving unit 124 typically includes an Integrated Circuit (IC), and has a fixed and unadjustable acquisition clock.

With reference to FIG. 2 again, the power supply circuit 140 may include: a main power supply sub-circuit 141, a high-voltage sub-circuit 142, and a low-voltage sub-circuit 143.

The main power supply sub-circuit 141 is respectively connected to the high-voltage sub-circuit 142 and the low-voltage sub-circuit 143, the high-voltage sub-circuit 142 is connected to the ultrasonic transmitting circuit 130 through a power cable, and the low-voltage sub-circuit 143 is connected to the ultrasonic receiving circuit 120 through a power cable. Specifically, the low-voltage sub-circuit 143 is connected to the power supply unit 125 through the power cable.

In an embodiment, the main power supply sub-circuit 141 may be a Direct Current-Direct Current (DC-DC) circuit.

With reference to FIG. 2 again, in an example, the main power supply sub-circuit 141 may output a voltage of ±15V to the high-voltage sub-circuit 142 and the low-voltage sub-circuit 143, the high-voltage sub-circuit 142 converts the voltage of ±15V into a high voltage of ±100V and outputs it to the ultrasonic transmitting circuit 130, and the low-voltage sub-circuit 143 converts the voltage of ±15V into a common voltage of 10V, 5V or 3.3V and outputs to the power supply unit 125.

FIG. 6 is a block diagram of another ultrasonic receiving circuit according to an embodiment of the present disclosure. As shown in FIG. 6, in the embodiment, the ultrasonic receiving circuit 120 may include: a data acquisition sub-circuit 125, an interpolation point determination sub-circuit 126 and a data compensation sub-circuit 127.

The data acquisition sub-circuit 125 may be configured to acquire array element data of each array element in the ultrasonic transducer array. The interpolation point determination sub-circuit 126 may be configured to determine one of the plurality of array elements as a reference array element and the other array elements except the reference array element among the plurality of array elements as to-be-compensated array elements, and determine interpolation points of the to-be-compensated array elements according to a scanning position of the array element data of the reference array element and an acquisition moment of each array element data. The data compensation sub-circuit 127 may be configured to perform data compensation on the interpolation points to obtain interpolation data.

In an embodiment, the data compensation sub-circuit 127 may be configured to determine interpolation data corresponding to an interpolation point according to a distance between a scanning position of the interpolation point and a scanning position of a point adjacent to the interpolation point and array element data of the adjacent point.

In an embodiment, the interpolation point determination sub-circuit 126 may be configured to determine the array element having the largest amount of data among the array elements as the reference array element. In other words, the interpolation point determination sub-circuit 126 takes the array element, which has the largest amount of array element data, among the plurality of array elements as the reference array element.

In an embodiment, the data acquisition sub-circuit 125 may be further configured to acquire acoustic-path data acquired by each array element in a plurality of initial scanning segments.

In an embodiment, in addition to the data acquisition sub-circuit 125, the interpolation point determination sub-circuit 126 and the data compensation sub-circuit 127, the ultrasonic receiving circuit 120 further includes a data segmentation circuit.

The data segmentation circuit may be configured to: determine a difference between two pieces of acoustic path data of each array element corresponding to each initial scanning segment and take the difference as an acoustic path difference of the array element in the initial scanning segment; determine a ratio of the acoustic path difference of each to-be-compensated array element to the acoustic path difference of the reference array element in each initial scanning segment; determine a change curve of the ratios of the acoustic path differences of each to-be-compensated array element with scan depths according to the ratios of the acoustic path differences of each to-be-compensated array element in the plurality of initial scanning segments; and segment a scan depth of each array element according to the change curve to obtain a plurality of compensation scanning segments. The initial scanning segment is a depth range formed with two adjacent acquisition points of acoustic path data as endpoints.

In an embodiment, the data segmentation circuit may be specifically configured to: determine the initial scanning segment corresponding to the ratio of the acoustic path difference which is smaller than an acoustic-path-difference threshold as a first scan depth range; determine the initial scanning segment corresponding to the ratio of the acoustic path difference which is greater than or equal to the acoustic-path-difference threshold as a second scan depth range; segment the first scan depth range with a first unit depth taken as an interval; and segment the second scan depth range with a second unit depth taken as an interval. The first unit depth is smaller than the second unit depth.

In an embodiment, the interpolation point determination sub-circuit 126 may be specifically configured to: according to a scanning position of array element data of the reference array element in each compensation scanning segment and the acquisition moment of each array element data, determine an interpolation point of the to-be-compensated array element in the same compensation scanning segment.

In an embodiment, the interpolation point determination sub-circuit 126 may be specifically configured to: for each to-be-compensated array element, according to a corresponding position of the array element data of the reference array element on the scanning line in each compensation scanning segment, determine a corresponding position of the to-be-compensated array element on the scanning line at a same acquisition moment, and take the corresponding position as an interpolation point of the to-be-compensated array element in the same compensation scanning segment.

In an embodiment, in addition to the data acquisition sub-circuit 125, the interpolation point determination sub-circuit 126 and the data compensation sub-circuit 127, the ultrasonic receiving circuit 120 further includes: an interpolation data correction circuit.

The interpolation data correction circuit may be configured to determine, after the interpolation data corresponding to the interpolation point is determined, a correction coefficient according to the determined interpolation data in the compensation scanning segment, and correct the interpolation data according to the correction coefficient.

It should be understood that the block diagrams of the systems or circuits shown in FIG. 1 to FIG. 6 do not make any limitation to the embodiments of the present disclosure.

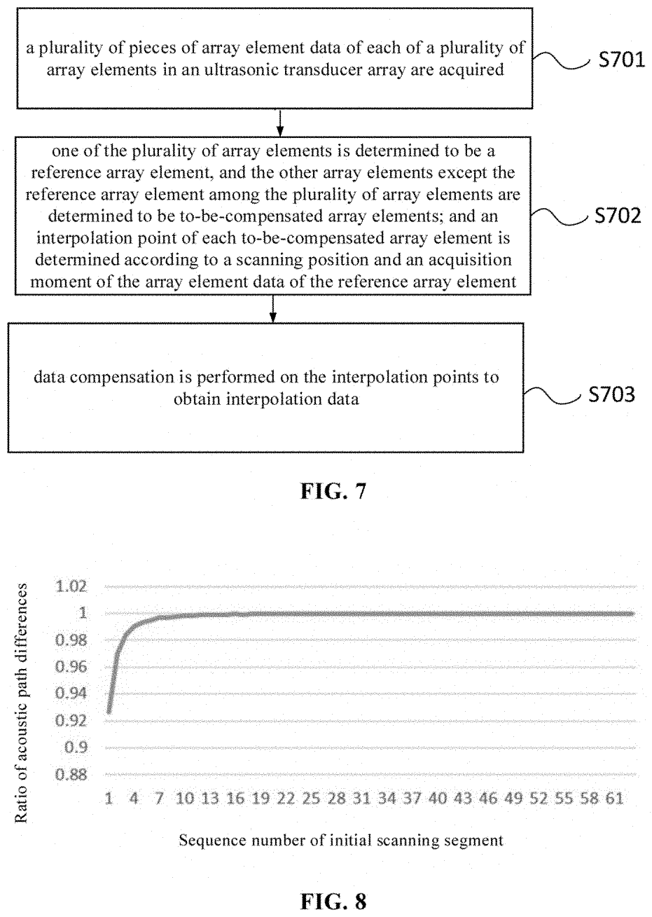

FIG. 7 is a flowchart illustrating a data processing method for the ultrasonic imaging system according to an embodiment of the present disclosure. The data processing method for the ultrasonic imaging system illustrated by FIG. 7 is applicable to data processing equipment. As shown in FIG. 7, in the present embodiment, the method includes steps S701 to S703.

At the step S701, a plurality of pieces of array element data of each of a plurality of array elements in an ultrasonic transducer array are acquired.

For each array element, each array element data of the array element is an ultrasonic echo signal acquired by the array element at an acquisition moment through a point on a scanning line.

At the step S702, one of the plurality of array elements is determined to be a reference array element, the other array elements except the reference array element are determined to be to-be-compensated array elements, and an interpolation point corresponding to each of the to-be-compensated array elements is determined according to a scanning position of each array element data of the reference array element and the acquisition moment of each array element data.

In an embodiment, the array element having the largest amount of array element data among the plurality of array elements may be determined to be the reference array element.

Specifically, the array element closest to the acquisition centerline among the plurality of array elements may be determined to be the reference array element, and the other array elements may be determined to be the to-be-compensated array elements. The reason is simple. For example, since the array elements vary in acoustic path to the acquisition centerline, the array element closest to the acquisition centerline acquires the largest amount of data under the condition of the same sampling rate. By performing data compensation on the other array elements by taking the array element closest to the acquisition centerline as a reference, each array element may retain data in basically the same amount as the data of the reference array element after the data compensation, which facilitates improvement on data comprehensiveness and accuracy of beam synthesis.

In an embodiment, after one of the plurality of array elements is determined to be the reference array element, and the other array elements except the reference array element are determined to be the to-be-compensated array elements, and before the interpolation point corresponding to each of the to-be-compensated array elements is determined according to the scanning position of each array element data of the reference array element and the acquisition moment of each array element data, the method may further include:

acquiring acoustic path data acquired by each array element in a plurality of initial scanning segments; determining a difference between two pieces of acoustic path data of each array element corresponding to each initial scanning segment and taking the difference as an acoustic path difference of the array element in the initial scanning segment; determining a ratio of the acoustic path difference of each to-be-compensated array element to the acoustic path difference of the reference array element in each initial scanning segment; determining a change curve of the ratios of the acoustic path differences of each to-be-compensated array element with scan depths according to the ratios of the acoustic path differences of each to-be-compensated array element in the plurality of initial scanning segments; and segmenting a scan depth of each array element according to the change curve to obtain a plurality of compensation scanning segments. In an embodiment, the initial scanning segment may be a depth range formed with two adjacent acquisition points of acoustic path data (i.e., focal points, as shown by the dots on the dashed line in FIG. 3) as endpoints.

In an embodiment, the scan depth represents a length of a scanning line (the dashed line in FIG. 3) corresponding to the array element, and a distance from each point (e.g., a focal point or a point between two focal points) on the scanning line to an intersection point of the scanning line and a plane to which the array element belongs is a scan depth value of the point. In an embodiment, the scanning position of the array element data represents a position of an acquisition point of the array element data on the scanning line, and may be represented by the scan depth value, and the acquisition point of the array element data may be any point on the scanning line.

In an example, if the ultrasonic probe is an 80-element convex array probe, acoustic path data of a part of the array elements are shown in Table 1. Considering the symmetry of the array elements, the acoustic path data of only four array elements (namely, an array element 1, an array element 2, an array element 3, and an array element 4) are listed in Table 1 as an example. The acoustic path data of different array elements are necessarily inconsistent when the same sampling rate is adopted, as shown in Table 1.

TABLE 1
Acoustic Paths of Different Array Elements

| Distance to | Array | Array | Array | Array |
|---|---|---|---|---|
| Focal | element 4 | element 3 | element 2 | element 1 |
| Point | (ns) | (ns) | (ns) | (ns) |
| 3mm | 3930 | 4063 | 4306 | 4628 |
| 6mm | 7810 | 7882 | 8021 | 8222 |
| 9mm | 11700 | 11751 | 11850 | 11996 |
| 12mm | 15594 | 15634 | 15712 | 15828 |
| 186mm | 35070 | 35091 | 35134 | 35197 |
| 189mm | 38966 | 38986 | 39025 | 39084 |
| 192mm | 42862 | 42880 | 42917 | 42973 |

In an embodiment, the sampling rate may represent the number of samples per second taken from a continuous signal to make a discrete signal, and is typically expressed in hertz (Hz).

With reference to the example illustrated by FIG. 3, the distance to focal point in Table 1 is a distance from the focal point (i.e., the acquisition point of acoustic path data) to the intersection point of the scanning line and the plane to which the array element belongs, that is, the scan depth value of the focal point. The adjacent distances to focal point in Table 1 correspond to adjacent focal points, and a depth range formed by taking every two adjacent focal points as endpoints is one initial scanning segment.

Taking the distances to focal point and the acoustic path data shown in Table 1 as an example, the first initial scanning segment is from 3 mm to 6 mm, and the two pieces of acoustic path data of the array element 1 corresponding to the first initial scanning segment are 4628 ns acquired at the distance to focal point of 3 mm and 8222 ns acquired at the distance to focal point of 6 mm respectively, so that the acoustic path difference of the array element 1 in such scan depth segment is 3594 ns; the two pieces of acoustic path data of the array element 2 corresponding to the first initial scanning segment are 4306 ns acquired at the distance to focal point of 3 mm and 8021 ns acquired at the distance to focal point of 6 mm respectively, so that the acoustic path difference is 3715 ns; and the acoustic path data of the array element 3 and the array element 4 corresponding to the first initial scanning segment are shown in Table 1, and the acoustic path differences are 3819 ns and 3880 ns respectively.

The second initial scanning segment is from 6 mm to 9 mm, the third initial scanning segment is from 9 mm to 12 mm, and so on. The acoustic path data of each array element corresponding to each initial scanning segment are shown in Table 1, the calculation of the acoustic path difference of each array element corresponding to each initial scanning segment is the same as that corresponding to the first initial scanning segment, and thus is not repeated.

Taking the array element 1 in Table 1 as an example, a ratio of the acoustic path difference of the array element 1 in each initial scanning segment to the acoustic path difference of the array element 4 in the same initial scanning segment is calculated, and then a change curve of the ratios of the acoustic path differences shown in FIG. 8 can be obtained. Sequence numbers of the initial scanning segments are taken as abscissas and the calculated ratios are taken as ordinates in the change curve in FIG. 8, the abscissa of 1 represents the first initial scanning segment, the abscissa of 4 represents the fourth initial scanning segment in FIG. 8, and so on; and then the scan depth may be re-segmented according to the change curve. Similarly, ratios of the acoustic path differences of the array element 2/3 to the array element 4 and change curves of the ratios may be calculated.

In an embodiment, segmenting the scan depth of the to-be-compensated array element according to the change curve includes:

determining the initial scanning segment corresponding to the ratio of the acoustic path differences which is smaller than an acoustic-path-difference threshold and taking the initial scanning segment as a first scan depth range; determining the initial scanning segment corresponding to the ratio of the acoustic path differences which is greater than or equal to the acoustic-path-difference threshold and taking the initial scanning segment as a second scan depth range; segmenting the first scan depth range with a first unit depth taken as an interval; and segmenting the second scan depth range with a second unit depth taken as an interval. The first unit depth is smaller than the second unit depth.

Taking the change curve in FIG. 8 as an example, it can be seen from the change curve in FIG. 8 that the ratio of the acoustic path difference of the array element 1 to the acoustic path difference of the array element 4 becomes closer to 1 as the scan depth is increased, that is, the acoustic path difference of the array element 1 and the acoustic path difference of the array element 4 are more and more consistent as the scan depth is increased. Therefore, the scan depth range may be segmented in such a way that the smaller scan depth range (that is, the range where the acoustic path differences between the two array elements are relatively large) is finely segmented by a smaller interval, and the larger scan depth range (that is, the range where the acoustic path differences between the two array elements are relatively small) is roughly segmented by a larger interval.

The acoustic-path-difference threshold may be set according to actual conditions, in the example illustrated by FIG. 8, the acoustic-path-difference threshold may be determined according to the trend of the change curve, for example, a value (e.g., 0.98 or 0.99) approaching to 1 may be set as the acoustic-path-difference threshold; and the first unit depth and the second unit depth may be set according to actual needs or empirical values, for example, the first unit depth may be set to 3 mm and the second unit depth may be set to 9 mm.

In connection with the example illustrated by Table 1, assuming that the ordinate value corresponding to the 15^{th} initial scanning segment (i.e., 42 mm-45 mm) is taken as the acoustic-path-difference threshold, the scan depths of 42 mm and below may be taken as the first scan depth range, and every 3 mm of the range is taken as one compensation scanning segment; and the scan depths above 42 mm may be taken as the second scan depth range, and every 9 mm of the range is taken as one compensation scanning segment.

In an embodiment, after the change curve as shown in FIG. 8 is obtained, the array element data of the to-be-compensated array element and the array element data of the reference array element may be segmented based on the scan depth in the following way:

determining a slope of each point on the change curve; determining a first slope range and a second slope range according to the slopes of all the points; segmenting a scan depth range corresponding to the first slope range by taking a first unit depth as an interval; and segmenting a scan depth range corresponding to the second slope range by taking a second unit depth as an interval. The slopes within the first slope range are all larger than those within the second slope range, and the first unit depth is smaller than the second unit depth.

A slope threshold may be set as a reference value when the first slope range and the second slope range are determined, and the slope threshold may be set according to actual conditions.

In an embodiment, determining the interpolation point of the to-be-compensated array element according to the scanning position of each array element data of the reference array element and the acquisition moment of each array element data includes: according to a scanning position of array element data of the reference array element in each compensation scanning segment and the acquisition moment of each array element data, determining an interpolation point of the to-be-compensated array element in the same compensation scanning segment.

In an embodiment, determining the interpolation point of the to-be-compensated array element in each compensation scanning segment according to the scanning position of the array element data of the reference array element in the same compensation scanning segment and the acquisition moment of each array element data includes: for each to-be-compensated array element, according to a corresponding position of the array element data of the reference array element on the scanning line (the dashed line in FIG. 3) in each compensation scanning segment, determining a corresponding position of the to-be-compensated array element on the scanning line at a same acquisition moment, and taking the corresponding position as the interpolation point of the to-be-compensated array element in the same compensation scanning segment.

In an example, when the array element 4 illustrated by Table 1 is taken as the reference array element, for the array element data Da received by the array element 4 in a certain compensation scanning segment, a scanning position A0 of Da on a scanning line corresponding to the array element 4 may be determined, so that a position Ax corresponding to the scanning position A0 on a scanning line of the array element 1 at the acquisition moment of Da may be determined, and Ax is the interpolation point of the array element 1 in the compensation scanning segment, that is, a position where interpolation is to be performed.

At the step S703, data compensation is performed on the interpolation point to obtain interpolation data.

In an embodiment, the interpolation data corresponding to the interpolation point is determined according to a distance between the scanning position of the interpolation point and a scanning position of a point adjacent to the interpolation point and array element data of the adjacent point.

In the present embodiment, the adjacent point may refer to a scanning position which is adjacent to the interpolation point on the same scanning line and where array element data is acquired, and an interpolation point usually have two adjacent points on the same scanning line.

In an embodiment, before the interpolation data corresponding to the interpolation point is determined, whether the interpolation point has array element data is first determined. When it is determined that the interpolation point has array element data, there is no need to determine the interpolation data and perform interpolation, so that unnecessary calculations may be reduced, and a data processing speed may be increased. When it is determined that the interpolation point has no array element data, the interpolation data corresponding to the interpolation point is determined according to the distance between the scanning positions of the interpolation point and the adjacent point and the array element data of the adjacent point, so that the data of the to-be-compensated array element may be compensated for so as to be aligned with the data of the reference array element, which facilitates beam synthesis.

In an example, if the interpolation point determined is A and the scanning positions adjacent to A are B and C respectively, the interpolation data Da (i.e., the array element data at the interpolation point A is to be compensated for) may be:

Da=K_{AC}×Db+K_{AB}×Dc Expression (1)

In the Expression (1), Db is the array element data at the position B, Dc is the array element data at the position C, K_{AC} is an interpolation coefficient determined based on L_{AC} (a distance between the interpolation point A and the position C), and K_{AB} is an interpolation coefficient determined based on LAB (a distance between the interpolation point A and the position B).

In an embodiment, K_{AC} and K_{AB} may be determined by:

$${K}_{AC} = \frac{{L}_{AC}}{{L}_{AC} + {L}_{AB}} {K}_{A B} = \frac{{L}_{AB}}{{L}_{AC} + {L}_{AB}} \qquad Expression ( 2 )$$

In the present embodiment, the way of determining K_{AC} and K_{AB} is not limited to the Expression (2), and K_{AC} and K_{AB} may be determined in other ways according to actual needs, for example, multiplying a certain coefficient on the basis of the Expression (2).

In an embodiment, a ratio of K_{AC} and K_{AB} may be equal to that of L_{AC} to L_{AB}.

In an embodiment, after the interpolation data corresponding to the interpolation point is determined, the method may further include: determining a correction coefficient according to the determined interpolation data in the compensation scanning segment; and correcting the determined interpolation data according to the correction coefficient to obtain corrected interpolation data.

In an example, the interpolation data obtained by the Expression (1) is corrected, and the obtained corrected interpolation data Da′ is:

Da′=K×(K_{AC}×Db+K_{AB}×Dc) Expression (3)

In the Expression (3), K is a correction coefficient, and the other parameters have the same meanings as above.

In an embodiment, the magnitude of the interpolation data may be changed by the correction coefficient, for example, the interpolation data may be expanded or reduced by a certain multiple. In an example, for example, the interpolation data calculated by the expression (1) is in thousands while the array element data of the reference array element is in tens, the correction coefficient should be set as a percentile (e.g., 0.01) in order to keep the order of magnitude of the interpolation data to be consistent with the array element data of the reference array element.

Thus, after the interpolation data Da is corrected by the correction coefficient K, the order of magnitude of the obtained corrected interpolation data Da′ is the same as that of the array element data of the reference array element, so that the orders of magnitude are kept uniform, and the interpolation data may be more accurate.

In an embodiment, the method may further include: storing the interpolation points and the interpolation data in a corresponding way for subsequent calling. The interpolation points and the interpolation data may be stored in one memory, or may be stored in a plurality of memories, which is not limited in the present disclosure.

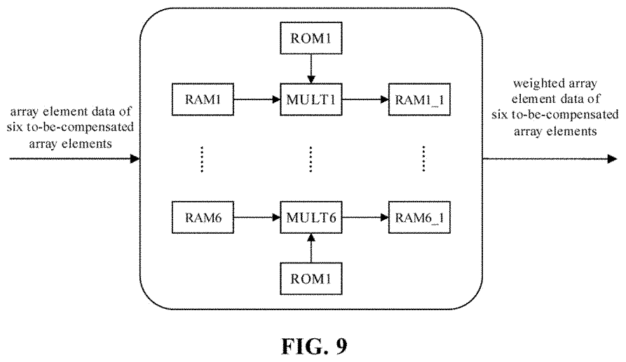

In an example, for the 8-element ultrasonic probe, the array element data of each to-be-compensated array element may be stored in one corresponding memory 211, the interpolation coefficient and the correction coefficient may also be stored in one corresponding memory 211, the multiplier circuit weights (for example, by the Expression (3)) the array element data, the interpolation coefficient and the correction coefficient of the to-be-compensated array element , and then stores the processed array element data of the to-be-compensated array element in one corresponding memory 211 for being called in subsequent data processing. A principle of a compensation process is shown in FIG. 9.

FIG. 9 is a schematic diagram illustrating a data compensation principle according to an embodiment of the present disclosure. As shown in FIG. 9, in the embodiment, RAM 1 to RAM6 are the memories for storing the array element data of the six to-be-compensated array elements respectively, ROM1 is the memory for storing the interpolation coefficients and the correction coefficients, MULT1 to MULT6 are the multiplier circuits for weighting the array element data of the six to-be-compensated array elements respectively, and RAM1_1 to RAM6_1 are configured to store the weighted array element data (i.e., the interpolation data) of the six to-be-compensated array elements respectively.

In another aspect, an embodiment of the present disclosure provides a computer storage medium having a computer program stored thereon. When the computer program is executed by a processor, the data processing method for the ultrasonic imaging system provided by the embodiments of the present disclosure is implemented.

In an embodiment, the computer storage medium may further store array element data of a plurality of array elements, and interpolation points and interpolation data obtained according to the data processing method for the ultrasonic imaging system provided by the embodiments of the present disclosure.

In an embodiment, the computer storage medium includes, but is not limited to, any type of disk (including a floppy disk, a hard disk, an optical disc, a Compact Disc Read-Only Memory (CD-ROM) and a magneto-optical disk), an ROM, a Random Access Memory (RAM), an Erasable Programmable Read-Only Memory (EPROM), an Electrically EPROM (EEPROM), a flash memory, and an a magnetic card or an optical card. That is, the storage medium includes any medium that can store or transmit information in a form readable by a device (e.g., a computer).

The computer storage medium provided by the embodiments of the present disclosure is applicable to the above data processing method for the ultrasonic imaging system and various implementations thereof, and the application of the computer storage medium is not described in detail here.

The technical solutions of the embodiments of the present disclosure can at least produce the following beneficial effects:

1) According to the embodiments of the present disclosure, on the basis that the reference array element is determined, the interpolation point of the to-be-compensated array element can be determined according to the reference array element, and the interpolation data of the to-be-compensated array element can be determined based on the interpolation point according to the positions of the adjacent points and the array element data of the adjacent points, so that compensation for the array element data of the to-be-compensated array element can be achieved, each to-be-compensated array element has the same amount of data as the reference array element within the same distance on a scanning line, and the array element data of each to-be-compensated array element can be aligned with that of the reference array element. Thus, the array element data of each array element can meet the requirement of beam synthesis, and the accuracy of ultrasonic imaging can be improved.

2) According to the embodiments of the present disclosure, the array element having the largest amount of data is selected as the reference array element, and data compensation is performed on the other array elements by taking the selected array element as a reference, so that more data can be retained by each array element after the compensation, thereby facilitate the improvement on the data comprehensiveness and accuracy of beam synthesis.

3) According to the embodiments of the present disclosure, the scan depth can be segmented to allow each array element data to be respectively compensated for based on each compensation scanning segment, which can, compared with a full-scanning-segment compensation method, effectively improve fineness of data compensation, thereby improving local definition of ultrasonic imaging.

4) According to the embodiments of the present disclosure, two segmentation processes are performed based on the acoustic path differences when the scan depth is segmented, two scan depth ranges having a great difference in change rule and different depths (one is deeper and the other one is shallower) are obtained in the first segmentation process, and the two scan depth ranges are further segmented separately in the second segmentation process, with the smaller scan depth range finely segmented and the larger scan depth range roughly segmented, so that the whole scan depth range are segmented more reasonably to refine granularity of data, and meanwhile, calculation processes can be simplified, and calculation amount can reduced to improve data processing efficiency.

It should be understood by those of ordinary skill in the art that the steps, measures and solutions in various operations, methods and flows discussed herein can be alternated, modified, combined or deleted. Moreover, other steps, measures and solutions in the various operations, methods and flows discussed herein can be alternated, modified, combined or deleted as well. Furthermore, the steps, measures and solutions in the prior art the same as those in the various operations, methods and flows discussed herein can be alternated, modified, combined or deleted as well.

In the description of the present disclosure, it should be understood that the terms “first” and “second” are only used to describe purposes and should not to be interpreted as indicating or implying relative importance or implicitly indicating the quantity of the defined technical features. Thus, the features defined by “first” and “second” can explicitly or implicitly include one or more of such features. In the description of the present disclosure, unless otherwise stated, the meaning of “a plurality” is two or more.

It should be understood that the steps in the flowcharts of the drawings, which are shown in orders as indicated by the arrows, are not necessarily performed in the orders as indicated by the arrows. Unless otherwise indicated herein, the orders in which the steps are performed are not limited, so that the steps can be performed in other orders. Moreover, at least a part of the steps in the flowcharts of the drawings can include a plurality of sub-steps or a plurality of stages, and those sub-steps or stages are not necessarily performed at the same time, but may be performed at different time. Moreover, those sub-steps or stages are not necessarily performed in a sequential order, and can be alternated with the other steps or at least a part of the sub-steps or stages of the other steps.

The above description is merely for some embodiments of the present disclosure. It should be noted that various changes and modifications can be made by those of ordinary skill in the art without departing from the principle of the present disclosure, and those changes and modifications should be considered to fall within the scope of the present disclosure.

1. A data processing method for an ultrasonic imaging system, comprising:
acquiring a plurality of pieces of array element data of each of a plurality of array elements in an ultrasonic transducer array;
determining one of the plurality of array elements to be a reference array element and the other array elements except the reference array element among the plurality of array elements to be to-be-compensated array elements;
determining interpolation points of the to-be-compensated array elements according to a scanning position and an acquisition moment of each array element data of the reference array element; and
performing data compensation on the determined interpolation points to obtain interpolation data.

2. The data processing method of claim 1, wherein the performing data compensation on the interpolation points to obtain the interpolated data comprises:
determining the interpolation data corresponding to the interpolation point according to a distance between a scanning position of the interpolation point and a scanning position of a point adjacent to the interpolation point and array element data of the adjacent point.

3. The data processing method of claim 2, after the interpolation data corresponding to the interpolation points are determined, further comprising:
determining a correction coefficient according to the interpolation data in a predetermined compensation scanning segment; and
correcting the interpolation data according to the correction coefficient.

4. The data processing method of claim 3, wherein the determining the correction coefficient according to the interpolation data in the predetermined compensation scanning segment comprises:
determining the correction coefficient according to order of magnitude of the interpolation data in the predetermined compensation scanning segment; and
the correcting the interpolation data according to the correction coefficient comprises:
correcting the interpolation data according to the correction coefficient to make order of magnitude of corrected interpolation data the same as that of the array element data of the reference array element.

5. The data processing method of claim 1, wherein the determining one of the plurality of array elements to be the reference array element comprises:
determining one of the plurality of array elements having the largest amount of array element data to be the reference array element.

6. The data processing method of claim 1, after the determining one of the plurality of array elements to be the reference array element and the other array elements except the reference array element among the plurality of array elements to be the to-be-compensated array elements, and before the determining the interpolation points of the to-be-compensated array elements according to the scanning position and the acquisition moment of each array element data of the reference array element, further comprising:
acquiring acoustic path data acquired by each of the plurality of array elements in a plurality of initial scanning segments;
determining a difference between two pieces of acoustic path data of each of the plurality of array elements corresponding to each initial scanning segment and taking the difference as an acoustic path difference of the array element in the initial scanning segment, with the initial scanning segment being a depth range formed with two adjacent acquisition points of acoustic path data as endpoints;
determining a ratio of the acoustic path difference of each of the to-be-compensated array elements to the acoustic path difference of the reference array element in each initial scanning segment;
according to the ratios of the acoustic path differences of each of the to-be-compensated array elements in the plurality of initial scanning segments, determining a corresponding change curve of the ratios of the acoustic path differences of the to-be-compensated array element with scan depths; and
segmenting a scan depth of each of the array elements according to the corresponding change curve to obtain a plurality of compensation scanning segments.

7. The data processing method of claim 6, wherein the segmenting the scan depth of the to-be-compensated array element according to the change curve comprises:
determining the initial scanning segment corresponding to the ratio of the acoustic path differences which is smaller than an acoustic-path-difference threshold and taking the initial scanning segment as a first scan depth range;
determining the initial scanning segment corresponding to the ratio of the acoustic path differences which is greater than or equal to the acoustic-path-difference threshold and taking the initial scanning segment as a second scan depth range;
segmenting the first scan depth range with a first unit depth taken as an interval; and
segmenting the second scan depth range with a second unit depth taken as an interval,
wherein the first unit depth is smaller than the second unit depth.

8. The data processing method of claim 6, wherein the determining the interpolation points of the to-be-compensated array elements according to the scanning position and the acquisition moment of each array element data of the reference array element comprises:
according to the scanning position and the acquisition moment of the array element data of the reference array element in each compensation scanning segment, determining the interpolation point of the to-be-compensated array element in the corresponding compensation scanning segment.

9. The data processing method of claim 8, wherein the determining the interpolation point of the to-be-compensated array element in the corresponding compensation scanning segment according to the scanning position and the acquisition moment of the array element data of the reference array element in each compensation scanning segment comprises:
for each of the to-be-compensated array elements, according to a corresponding position of the array element data of the reference array element on a scanning line in each compensation scanning segment, determining a corresponding position of the to-be-compensated array element on the scanning line at a same acquisition moment, and taking the corresponding position as the interpolation point of the to-be-compensated array element in the corresponding compensation scanning segment.

10. An ultrasonic imaging system, comprising: an ultrasonic transducer array and an ultrasonic receiving circuit;
the ultrasonic transducer array comprises a plurality of array elements; and
the ultrasonic receiving circuit is connected to and communicates with each of the plurality of array elements, and is configured to receive ultrasonic echo signals acquired by the plurality of array elements as array element data and perform the data processing method for the ultrasonic imaging system of claim 1.

11. The ultrasonic imaging system of claim 10, further comprising: an ultrasonic transmitting circuit and a power supply circuit;
the ultrasonic transmitting circuit is connected to and communicates with each of the plurality of array elements and is configured to generate electrical signals and excite the plurality of array elements with the electrical signals to transmit ultrasonic waves; and
the power supply circuit is electrically connected to the ultrasonic receiving circuit and the ultrasonic transmitting circuit respectively, and is configured to supply power to the ultrasonic receiving circuit and the ultrasonic transmitting circuit.

12. The ultrasonic imaging system of claim 10, wherein the ultrasonic receiving circuit comprises:
a memory;
a processor electrically connected to the memory; and
the memory has a computer program stored thereon, and the computer program is executed by the processor to implement the data processing method for the ultrasonic imaging system.

13. The ultrasonic imaging system of claim 10, wherein the ultrasonic receiving circuit comprises:
a data acquisition sub-circuit configured to acquire array element data of each of the plurality of array elements;
an interpolation point determination sub-circuit configured to determine one of the plurality of array elements to be a reference array element and the other array elements except the reference array element among the plurality of array elements to be to-be-compensated array elements, and determine interpolation points of the to-be-compensated array elements according to scanning positions and acquisition moments of the array element data of the reference array element; and
a data compensation sub-circuit configured to perform data compensation on the interpolation points to obtain interpolation data.

14. A computer storage medium having a computer program stored thereon, wherein, when the computer program is executed by a processor, the data processing method for the ultrasonic imaging system of claim 1 is implemented.

15. The data processing method of claim 4, wherein the determining one of the plurality of array elements to be the reference array element comprises:
determining one of the plurality of array elements having the largest amount of array element data to be the reference array element.

16. The data processing method of claim 15, after the determining one of the plurality of array elements to be the reference array element and the other array elements except the reference array element among the plurality of array elements to be the to-be-compensated array elements, and before the determining the interpolation points of the to-be-compensated array elements according to the scanning position and the acquisition moment of each array element data of the reference array element, further comprising:
acquiring acoustic path data acquired by each of the plurality of array elements in a plurality of initial scanning segments;
determining a difference between two pieces of acoustic path data of each of the plurality of array elements corresponding to each initial scanning segment and taking the difference as an acoustic path difference of the array element in the initial scanning segment, with the initial scanning segment being a depth range formed with two adjacent acquisition points of acoustic path data as endpoints;
determining a ratio of the acoustic path difference of each of the to-be-compensated array elements to the acoustic path difference of the reference array element in each initial scanning segment;
according to the ratios of the acoustic path differences of each of the to-be-compensated array elements in the plurality of initial scanning segments, determining a corresponding change curve of the ratios of the acoustic path differences of the to-be-compensated array element with scan depths; and
segmenting a scan depth of each of the array elements according to the corresponding change curve to obtain a plurality of compensation scanning segments.

17. The data processing method of claim 16, wherein the segmenting the scan depth of the to-be-compensated array element according to the change curve comprises:
determining the initial scanning segment corresponding to the ratio of the acoustic path differences which is smaller than an acoustic-path-difference threshold and taking the initial scanning segment as a first scan depth range;
determining the initial scanning segment corresponding to the ratio of the acoustic path differences which is greater than or equal to the acoustic-path-difference threshold and taking the initial scanning segment as a second scan depth range;
segmenting the first scan depth range with a first unit depth taken as an interval; and
segmenting the second scan depth range with a second unit depth taken as an interval,
wherein the first unit depth is smaller than the second unit depth.

18. The data processing method of claim 16, wherein the determining the interpolation points of the to-be-compensated array elements according to the scanning position and the acquisition moment of each array element data of the reference array element comprises:
according to the scanning position and the acquisition moment of the array element data of the reference array element in each compensation scanning segment, determining the interpolation point of the to-be-compensated array element in the corresponding compensation scanning segment.

19. The data processing method of claim 18, wherein the determining the interpolation point of the to-be-compensated array element in the corresponding compensation scanning segment according to the scanning position and the acquisition moment of the array element data of the reference array element in each compensation scanning segment comprises:
for each of the to-be-compensated array elements, according to a corresponding position of the array element data of the reference array element on a scanning line in each compensation scanning segment, determining a corresponding position of the to-be-compensated array element on the scanning line at a same acquisition moment, and taking the corresponding position as the interpolation point of the to-be-compensated array element in the corresponding compensation scanning segment.

20. The ultrasonic imaging system of claim 11, wherein the ultrasonic receiving circuit comprises:
a memory;
a processor electrically connected to the memory; and
the memory has a computer program stored thereon, and the computer program is executed by the processor to implement the data processing method for the ultrasonic imaging system.
